(19) United States
(12) Patent Application Publication
(10) Pub. No.: US 2009/0176855 A1
(43) Pub. Date: Jul. 9, 2009

(54) TREATMENT OF BIPOLAR DISORDERS AND ASSOCIATED SYMPTOMS

(75) Inventors: Erik Ho Fong Wong, Ann Arbor, MI (US); John Panagides, West Nyack, NY (US); Jacques Schipper, Oss (NL)

Correspondence Address: ORGANON USA, INC.;c/o Schering-Plough Corporation, 2000 Galloping Hill Road, Mail Stop: K-6-1, 1990, Kenilworth, NJ, 07033, US

(73) Assignee: N.V. Organon

(21) Appl. No.: 12/405,689

(22) Filed: Mar. 17, 2009

Related U.S. Application Data
(62) Division of application No. 11/247,342, filed on Oct. 11, 2005.
(60) Provisional application No. 60/619,274, filed on Oct. 15, 2004.
(60) Provisional application No. 60/620,178, filed on Oct. 19, 2004.

(51) Int. Cl. A61K 31/407; A61P 25/18

(52) U.S. Cl. 514/410

(57) ABSTRACT

The present invention relates to a method and kits for treating bipolar disorder in a mammal, including a human, the treatments including treatment of bipolar disorder including rapid-cycling, treatment of symptoms of bipolar disorder including acute mania or hypomania and depression, and episodes or occurrences including both acute mania or hypomania and depression; treatment for effecting mood stabilization; treatment for preventing relapse into bipolar episodes, and for the treatment of suicidal thoughts and tendencies associated with bipolar disorder, comprising administering to said mammal an effective amount of a compound of Formula

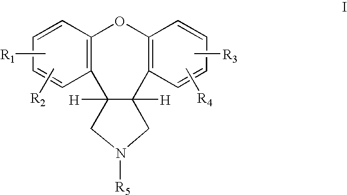

or a pharmaceutically acceptable salt, solvate, hydrate or optical isomer thereof.

Priority of this application is based on U.S. Provisional Application Ser. No. 60/619,274, filed Oct. 15, 2004 and U.S. Provisional Application Ser. No. 60/620,178, filed Oct. 19, 2004.

### FIELD OF THE INVENTION

The present invention relates to the treatment of bipolar disorder in a mammal, including a human. More specifically, the present invention is directed to the treatment in a mammal, including a human, of bipolar disorder including the rapid-cycling form, and for the treatment of symptoms of bipolar disorder, such symptoms including acute mania or hypomania, depression, and episodes or occurrences including both acute mania or hypomania and depression. The present invention is also directed to a treatment method for effecting mood stabilization in a person afflicted with bipolar disorder. The present invention further relates to a method of preventing relapse in mood disturbances including acute mania or hypomania and depression into bipolar episodes in a person afflicted with bipolar disorder. The present invention is further directed to treating suicidal thoughts and tendencies in a person afflicted with bipolar disorder. The present invention is further directed to the treatment of bipolar disorders with at least one other co-morbid or concomitant disease, condition, or disorder. The present invention also relates to new therapeutic uses for trans-5-chloro-2-methyl-2,3,3a, 12b-tetrahydro-1H-dibenz[2,3:6,7]oxepino[4,5-c]pyrrole, also known as asenapine, as defined below.

### BACKGROUND OF THE INVENTION

The compounds of Formula I of this invention are disclosed in U.S. Pat. Nos. 4,145,434 and 5,763,476. Certain treatments for these compounds are also disclosed in U.S. Pat. Nos. 4,145,434 and 5,763,476. The patents listed in this paragraph are incorporated by reference in their entireties into the present disclosure.

### SUMMARY OF THE INVENTION

The present invention relates to the use of compounds of the Formula I, as defined below, in methods for the treatment of bipolar disorder in a mammal, including a human. Specifically, the present invention is directed to a method for the treatment in a mammal, including a human, of bipolar disorder including rapid-cycling bipolar disorder, a method for the treatment of symptoms of bipolar disorder, such symptoms selected from the group consisting of acute mania, hypomania, depression and episodes or occurrences including both acute mania or hypomania and depression; a method for a treatment that effects mood stabilization in a person afflicted with bipolar disorder; a method for a treatment that prevents relapse in mood disturbances including acute mania or hypomania and depression in a person afflicted with bipolar disorder; a method for the treatment of suicidal thoughts and tendencies in a person afflicted with bipolar disorder; a method for treatment of bipolar disorders with at least one co-morbid or concomitant disease, condition, or disorder. Said condition, disease, or disorder concomitant with bipolar disorder includes but is not limited to, depression melancholia, fatigue, personality disorders including avoidant personality disorder, borderline personality disorder, schizotypal personality disorder, and anxious personality disorder, aggressive disorders including intermittent explosive disorder and organic personality syndrome, oppositional defiant disorder, atypical cycloid psychoses, motility psychosis, confussional psychosis, anxiety-blissfulness psychosis, dementia and delirium, such treatments comprising administering a pharmaceutically effective amount of a compound of Formula I:

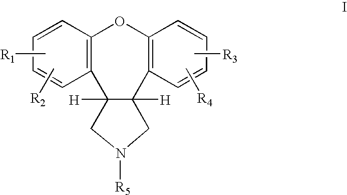

or a pharmaceutically acceptable salt, solvate, hydrate or optical isomer thereof, wherein R₁, R₂, R₃, and R₄ represent a member selected from the group consisting of hydrogen, hydroxy, halogen, C₁-C₆ alkyl, C₁-C₆ alkoxy, C₁-C₆ alkylthio, and trifluoromethyl; and
R₅ represents hydrogen, C₁-C₆ alkyl or aralkyl having from 7 to 10 carbon atoms.

One aspect of the present invention is directed to a method for the treatment in a mammal, including a human, of bipolar disorder including rapid-cycling bipolar disorder, a method for the treatment of symptoms of bipolar disorder, such symptoms selected from the group consisting of acute mania or hypomania, and depression and episodes or occurrences including both acute mania a hypomania and depression; a method for a treatment that effects mood stabilization in a person afflicted with bipolar disorder; a method for a treatment that prevents relapse into bipolar episodes in a person afflicted with bipolar disorder; a method for the treatment of suicidal thoughts and tendencies in a mammal afflicted with bipolar disorder; such treatments comprising administering to said mammal an effective amount of asenapine: trans-5-chloro-2-methyl-2,3,3a, 12b-tetrahydro-1H-dibenz[2,3:6,7]oxepino[4,5-c]pyrrole or a pharmaceutically acceptable salt thereof.

The term “asenapine”, as used herein, unless otherwise indicated, encompasses the free base of the compound asenapine (named in the preceding paragraph) and all pharmaceutically acceptable salts, solvates, hydrates, and optical isomers thereof. Asenapine is also known in the art as Org 5222.

Pharmaceutically acceptable addition salts include, but are not limited to, salts of the compounds of Formula I, such as maleate, mesylate, esylate, and hydrochloride, among others, and may also include polymorphic forms of such salts.

In yet another aspect of the present invention, the treatments described above improve the condition of a person afflicted with bipolar disorder, or symptoms associated with bipolar disorder as described above, within about 96 hours from the first administration of a compound of formula I, such as for example, asenapine. However, such improvements can be realized more rapidly, that is within about 24 to about 96 hours after administering a compound of formula I, such as for example, asenapine.

The psychiatric disorders and conditions referred to herein are known to those of skill in the art and are defined in art-recognized medical texts such as the Diagnostic and Statistical Manual of Mental Disorders, Fourth Edition, American Psychiatric Association, 1994 (DSM-IV), which is incorporated herein by reference in its entirety.

The term “treating”, as used herein, refers to reversing, alleviating, inhibiting the progress of, or preventing the reoccurrence of or preventing the disorder or condition to which such term applies, or one or more symptoms of such disorder or condition. The term “treatment”, as used herein, refers to the act of treating, as “treating” is defined immediately above. The terms “treat”, “treatment”, and “treating” include preventative (e.g., prophylactic) and palliative treatment or the act of providing preventative or palliative treatment.

The phrase “a patient in need thereof” is a patient who has or is at risk of having a condition as described herein.

The term “patient” means animals, particularly mammals. Preferred patients are humans.

The term “pharmaceutically effective amount”, as used herein, refers to an amount of the compound sufficient to treat, in a mammal, including a human bipolar disorder, symptoms of bipolar disorder including acute mania or hypomania and depression or combination thereof; to effect mood stabilization; to prevent relapse into bipolar episodes; and to a treat suicidal thoughts and tendencies.

As used herein, the term “effective amount” means an amount of compound that is capable of treating the described conditions. The specific dose of a compound administered according to this invention will, of course, be determined by the particular circumstances surrounding the case including, for example, the compound administered, the route of administration and the severity of the condition being treated.

As provided in the DSM-IV, the specifier of bipolar disorder with rapid cycling can be applied to Bipolar I Disorder or Bipolar II Disorder. The essential feature of a rapid-cycling Bipolar Disorder is the occurrence of four or more mood episodes during the previous 12 months.

The “symptoms of bipolar disorder selected from the group consisting of acute mania and depression” refer to, respectively, one or more symptoms that may be associated with a manic episode or a depressive episode, as the case may be, of bipolar disorder.

“Mood stabilization”, as used herein, refers to the suppression of manic symptoms and depressive symptoms in order to maintain a euthymic state in the subject of the treatment.

As used herein, the term “relapse prevention” refers to preventing the recurrence of a kind of episode in a subject who previously experienced at least one of that same kind of episode. An example of “relapse prevention” is preventing a recurrence of a manic episode in a subject who previously experienced one or more manic episodes.

The treatment of “suicidal thoughts and tendencies” refers to the suppression of suicidal ideation in a subject afflicted with bipolar disorder, with the further goal of suppressing suicide attempts.

By an aralkyl group is preferably meant a phenylalkyl group with 7-10 carbon atoms, such as benzyl, phenylethyl, phenylpropyl or 1-methylphenylethyl.

Also provided by the present invention are kits for use in treating bipolar disorders, the kits comprising:

A) a pharmaceutical composition comprising a compound of Formula I

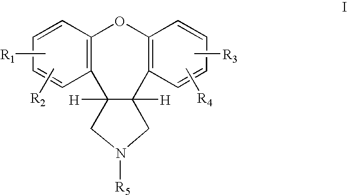

or a pharmaceutically acceptable salt or optical isomer thereof, wherein R₁, R₂, R₃, and R₄ represent a Member selected from the group consisting of hydrogen, hydroxy, halogen, C₁-C₆ alkyl, C₁-C₆ alkoxy, C₁-C₆ alkylthio, and trifluoromethyl;
R₅ represents hydrogen, C₁-C₆ alkyl or aralkyl having from 7 to 10 carbon atoms; and

B) instructions for administering the pharmaceutical composition comprising a compound of Formula I or a pharmaceutically acceptable salt or optical isomer thereof to a patient in need thereof to treat bipolar disorders.

### DETAILED DESCRIPTION OF THE INVENTION

Pharmaceutically-acceptable acid addition salts include, but is not limited to, salts such as the hydrochloride, hydrobromide, sulfate, hydrogen sulfate, phosphate, hydrogen phosphate, dihydrogenphosphate, acetate, succinate, tartrate, citrate, methanesulfonate (mesylate) and p-toluenesulfonate (tosylate) salts.

The pharmaceutically acceptable acid addition salts of the compounds of this invention may be formed of the compound itself, or of any of its esters, and include the pharmaceutically acceptable salts that are often used in pharmaceutical chemistry. For example, salts may be formed with inorganic or organic acids such as hydrochloric acid, hydrobromic acid, hydroiodic acid, sulfonic acids including such agents as naphthalenesulfonic, methanesulfonic and toluenesulfonic acids, sulfuric acid, nitric acid, phosphoric acid, tartaric acid, pyrosulfuric acid, metaphosphoric acid, succinic acid, formic acid, phthalic acid, lactic acid and the like, most preferably with hydrochloric acid, citric acid, benzoic acid, maleic acid, acetic acid or propionic acid.

The salts of basic compounds can be formed by reacting the compound with a suitable acid. The salts are typically formed in high yields at moderate temperatures, and often are prepared by isolating the compound from a suitable acidic wash as the final step of the synthesis. The salt-forming acid is dissolved in an appropriate organic solvent, or aqueous organic solvent, such as an alkanol, ketone or ester. On the other hand, if a compound is desired in the free base form, it can be isolated from a basic final wash step. A technique for preparing hydrochlorides is to dissolve the free base in a suitable solvent and dry the solution thoroughly, as over molecular sieves, before bubbling hydrogen chloride gas through it. It will also be recognized that it is possible to administer amorphous forms of the compounds.

One of ordinary skill in the art will recognize that certain compounds of this invention will contain one or more atoms which may be in a particular stereochemical, tautomeric, or geometric configuration, giving rise to stereoisomers, tautomers and configurational isomers. All such tautomers and isomers and mixtures thereof are included in this invention. Hydrates and solvates of the compounds of this invention are also included.

The subject invention also includes isotopically-labeled compounds, which are structurally identical to those disclosed above, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, sulfur, fluorine and chlorine, such as ²H, ³H, ¹³C, ¹⁴C, ¹⁵N, ¹⁸O, ¹⁷O, ³¹P, ³²P, ³⁵S, ¹⁸F and ³⁶Cl, respectively. Compounds of the present invention, prodrugs thereof, and pharmaceutically acceptable salts of said compounds and of said prodrugs which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, may afford certain therapeutic advantages resulting from greater metabolic stability, for example increased in vivo half-life or reduced dosage requirements and, hence, may be preferred in some circumstances. Isotopically labeled compounds of this invention and prodrugs thereof can generally be prepared by carrying out known or referenced procedures and by substituting a readily available isotopically labeled reagent for a non-isotopically labeled reagent.

Those of ordinary skill in the art will recognize that physiologically active compounds which have accessible hydroxy groups can be administered in the form of pharmaceutically acceptable esters. The compounds of this invention can be effectively administered as an ester, formed on the hydroxy groups. It is possible to adjust the rate or duration of action of the compound by appropriate choices of ester groups.

The dose of a compound of this invention to be administered to a subject is rather widely variable and subject to the judgement of the attending physician. It should be noted that it may be necessary to adjust the dose of a compound when it is administered in the form of a salt, such as a laureate, the salt forming moiety of which has an appreciable molecular weight.

The following dosage amounts are for an average human subject having a weight of about 65 kg to about 70 kg. One skilled in the art will readily be able to determine the dosage amount required for a subject whose weight falls outside the 65 kg to 70 kg range, based upon the medical history of the subject. All doses set forth herein are daily doses of the free base or acid forms. Calculation of the dosage amount for other forms of the free base or acid forms such as salts or hydrates is easily accomplished by performing a simple ratio relative to the molecular weights of the species involved.

The general range of effective administration rates of the compounds of Formula I is from about 0.1 mg/day to about 100 mg/day. Of course, it is often practical to administer the daily dose of a compound in portions, at various hours of the day. However, in any given case, the amount of compound administered will depend on such factors as the potency of the specific compound, the solubility of the compound, the formulation used and the route of administration.

When an active compound of this invention is to be used in a human subject to treat psychiatric conditions whose manifestations include psychiatric symptoms or behavioral disturbance, the prescribing physician will normally determine the daily dosage. Moreover, the dosage will vary according to the age, weight and response of the individual patient as well as the severity of the patient's symptoms. However, in most instances, an effective amount for treating the psychiatric conditions described herein, will be a daily dosage in the range from about 0.5 to about 500 mg, more specifically about 10 mg a day to about 200 mg a day, relatively more specifically about 5 mg a day to about 10 mg a day, in single or divided doses, orally or parenterally. In some instances it may be necessary to use dosages outside these limits.

The compounds of formula I can be prepared by one or more of the synthetic methods described and referred to in U.S. Pat. Nos. 4,145,434 and 5,763,476. U.S. Pat. Nos. 4,145,434 and 5,763,476 are incorporated herein by reference in their entireties. The compound asenapine: trans-5-chloro-2-methyl-2,3,3a, 12b-tetrahydro-1H-dibenz[2,3:6,7]oxepino[4,5-c]pyrrole can be prepared by one or more of the synthetic methods described and referred to in U.S. Pat. No. 4,145,434 and is incorporated herein by reference in its entirety.

Compounds of formula I, and their pharmaceutically acceptable salts (referred to collectively hereinafter, as “the active compounds of this invention”), can be administered to a human subject either alone, or, preferably, in combination with pharmaceutically acceptable carriers or diluents, in a pharmaceutical composition. Such compounds can be administered sublingually, buccally, or supralingually. See, for example. U.S. Pat. No. 5,763,476.

Additionally, in a pharmaceutical composition comprising an active compound of this invention, the weight ratio of active ingredient to carrier will normally be in the range from 1:6 to 2:1, and preferably 1:4 to 1:1. However, in any given case, the ratio chosen will depend on such factors as the solubility of the active component, the dosage contemplated and the precise route of administration.

For sublingual, buccal, and supralingual use in treating psychiatric conditions whose manifestations include psychiatric symptoms or behavioral disturbance, the active compounds of this invention can be administered, for example, in the form of tablets or lozenge, or as an aqueous solution or suspension. In the case of tablets for oral use, carriers that can be used include lactose and cornstarch, and lubricating agents, such as magnesium stearate, can be added. For oral administration in capsule form, useful diluents are lactose and dried cornstarch. When aqueous suspensions are required for oral use, the active ingredient can be combined with emulsifying and suspending agents. If desired, certain sweetening and/or flavoring agents can be added. For intramuscular, parenteral and intravenous use, sterile solutions of the active ingredient can be prepared, and the pH of the solutions should be suitably adjusted and buffered. For intravenous use, the total concentration of solutes should be controlled to render the preparation isotonic.

In an embodiment the pharmaceutical compositions of the invention are tablets or lozenges, which comprise a rapidly disintegrating composition of a pharmaceutically acceptable water-soluble or water-dispersible carrier material. Tablets and lozenges comprising a rapidly disintegrating composition of a pharmaceutically acceptable water-soluble or water-dispersible carrier material are known in the art, for example as disclosed in U.S. Pat. No. 4,371,516. Such tablets may be prepared by freeze-drying of an aqueous solution comprising trans-5-chloro-2-methyl-2,3,3a, 12b-tetrahydro-1H-dibenz[2,3:6,7]oxepino[4,5-c]pyrrole, a water-soluble or water-dispersible carrier material and, optionally, pharmaceutically acceptable excipients. Such excipients are known in the art, see for instance Remington's Pharmaceutical Sciences, 18th Edition (Ed. A. R. Genaro), 1990, pp 1635-1638, and are commonly used in pharmaceutical compositions, for instance surfactants, colouring agents, flavouring agents, preservatives and the like. The water-soluble or water-dispersible carrier material is preferably water-soluble. Suitable water-soluble carrier materials are (poly)saccharides like hydrolyzed dextran, dextrin, mannitol, and alginates, or mixtures thereof, or mixtures thereof with other carrier materials like polyvinylalcohol, polyvinylpyrrolidine and water-soluble cellulose derivatives, like hydroxypropyl cellulose.

In an embodiment, the carrier material is gelatin, especially partially hydrolyzed gelatin. The partially hydrolyzed gelatin can be prepared by heating of a solution of gelatin in water, for example in an autoclave at about 120° C. for up to 2 hours. The hydrolyzed gelatin is used in concentrations of about 1 to 6% (w/v), and preferably in concentrations of about 2 to 4% (w/v).

The dosage forms of the composition of the invention, i.e. tablets or lozenges, can be prepared by methods known in the art. For example, according to a method as disclosed in British Patent 2,111,423, an aqueous composition comprising a predetermined amount of trans-5-chloro-2-methyl-2,3,3a, 12b-tetrahydro-1H-dibenz[2,3:6,7]oxepino[4,5-c]pyrrole, a pharmaceutically acceptable water-soluble or water-dispersible carrier material and optionally pharmaceutically acceptable auxiliaries and excipients, is transferred into a mold, after which the composition is frozen and the solvent is sublimed, preferably by freeze-drying. The composition preferably contains a surfactant, for example Tween 80 (polyoxyethylene (20) sorbitan mono-oleate), which may help to prevent the freeze-dried product from sticking to the surface of the mold.

The mold may comprise a series of cylindrical or other shape depressions, each having a size corresponding to the desired size of the dosage form. Alternatively, the mold may have a larger size than the desired size of the dosage form, and after the contents are freeze-dried the product can be cut into the desired size. Preferably the dosage form is freeze-dried in the form of a lyosphere, which is a freeze-dried spherical-shaped droplet containing the active ingredient.

A mold would correspond to a depression in a sheet of film material, as for example disclosed in U.S. Pat. No. 4,305,502 and U.S. Pat. No. 5,046,618. The film material may be similar to that employed in conventional blister packs.

Each dosage form of the pharmaceutical composition of the present invention comprises one dosage unit of trans-5-chloro-2-methyl-2,3,3a, 12b-tetrahydro-1H-dibenz[2,3:6,7]oxepino[4,5-c]pyrrole as active ingredient. A dosage unit may contain between 0.005 mg and 20 mg of the active ingredient. Preferably the dosage unit contains 5-10 mg of trans-5-chloro-2-methyl-2,3,3a, 12b-tetrahydro-1H-dibenz[2,3:6,7]oxepino[4,5-c]pyrrole.

The present invention also provides kits for use to treat bipolar disorders.

The kits comprise: A) a pharmaceutical composition comprising a compound of Formula I or a pharmaceutically acceptable salt or optical isomer thereof; and B) instructions describing a method of using the pharmaceutical composition to treat bipolar disorder. In an embodiment of the kits, the compound is 5-chloro-2-methyl-2,3,3a, 12b-tetrahydro-1H-dibenz[2,3:6,7]oxepino[4,5-c]pyrrole, or a pharmaceutically acceptable salt thereof, or trans-5-chloro-2-methyl-2,3,3a, 12b-tetrahydro-1H-dibenz[2,3:6,7]oxepino[4,5-c]pyrrole, or a pharmaceutically acceptable salt thereof.

A “kit” as used in the instant application includes a container for containing the pharmaceutical compositions and may also include divided containers such as a divided bottle or a divided foil packet. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a written memory aid, where the written memory aid is of the type containing information and/or instructions for the physician, pharmacist or subject, e.g., in the form of numbers next to the tablets or capsules whereby the numbers correspond with the days of the regimen which the tablets or capsules so specified should be ingested or a card which contains the same type of information. Another example of such a memory aid is a calendar printed on the card e.g., as follows “First Week, Monday, Tuesday,” . . . etc. . . . “Second Week, Monday, Tuesday, . . . ” etc. Other variations of memory aids will be readily apparent. A “daily dose” can be a single tablet or capsule or several tablets or capsules to be taken on a given day.

Another specific embodiment of a kit is a dispenser designed to dispense the daily doses one at a time. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

### EXAMPLES

The invention is illustrated by the following Examples.

### Example 1

### a: Preparation of Hydrolyzed Gelatin (3% w/v)

Gelatin (30 g) was dissolved in 1 l of distilled water under heating and constant stirring. The resulting solution was autoclaved at 121° C. (10⁵ Pa) for one hour, upon which the solution was allowed to cool to room temperature to give hydrolyzed gelatin (3% w/v).

### b: Preparation of a Solid Pharmaceutical Dosage Form

A sheet of polyvinyl chloride (PVC) containing cylindrical depressions was cooled with solid carbon dioxide. 0.2 g of Org 5222, 5-chloro-2-methyl-2,3,3a, 12b-tetrahydro-1H-dibenz[2,3:6,7]oxepino[4,5-c]pyrrole maleate (1:1) were dissolved in 1 l of hydrolyzed gelatin under mixing. While mixing was continued, in each of the depressions 0.5 ml of the solution were placed. When the contents of the depressions were frozen, the PVC sheet was placed in a freeze-drying system. An aluminum foil was finally sealed to the sheet so as to close off the depressions containing the freeze-dried pharmaceutical dosage forms. Each depression contains a pharmaceutical unit dosage comprising 0.10 mg of 5-chloro-2-methyl-2,3,3a, 12b-tetrahydro-1H-dibenz[2,3:6,7]oxepino[4,5-c]pyrrole maleate (1:1).

### Example 2

In a manner as described in Example 1b a pharmaceutical composition was prepared comprising:

0.2 g of 5-chloro-2-methyl-2,3,3a, 12b-tetrahydro-1H-dibenz[2,3:6,7]oxepino[4,5-c]pyrrole maleate (1:1) (Org 5222), 0.50 g of Tween 80 (polyoxyethylene (20) sorbitan mono-oleate, 30 g of sucrose and 1 l of hydrolyzed gelatin (3% w/v).

### Example 3

In a manner as described in Example 1b a pharmaceutical composition was prepared comprising:

2 g of 5-chloro-2-methyl-2,3,3a, 12b-tetrahydro-1H-dibenz[2,3:6,7]oxepino[4,5-c]pyrrole maleate (1:1) (Org 5222), 0.50 g of Tween 80 (polyoxyethylene (20) sorbitan mono-oleate, 30 g of sucrose and 1 l of hydrolyzed gelatin (3% w/v), 1 l of hydrolyzed gelatin (3% w/v).

### Example 4

A pharmaceutical composition was prepared comprising:

0.2 g of 5-chloro-2-methyl-2,3,3a, 12b-tetrahydro-1H-dibenz[2,3:6,7]oxepino[4,5-c]pyrrole maleate (1:1) (Org 5222), 17 g of sodium alginate, 35 g of dextran (MW approx. 40.000), 17.5 g of dextrose, and distilled water to a volume of 1 l, which composition was freeze-dried into unit dosage forms.

### Example 5

A pharmaceutical composition was prepared comprising:

0.4 g of 5-chloro-2-methyl-2,3,3a, 12b-tetrahydro-1H-dibenz[2,3:6,7]oxepino[4,5-c]pyrrole maleate (1:1) (Org 5222), 50 g of dextrin, 0.20 g of Tween 80 (polyoxyethylene (20) sorbitan mono-oleate, 30 g of polyvinylpyrrolidine and distilled water to a volume of 1 l, which composition was freeze-dried into unit dosage forms.

### Example 6

Lyospheres were prepared by dissolving 138.9 g of sucrose, 40.8 g of sodium citrate, and 111 mg of polysorbate 20 in 300 ml of distilled water, adjusting the pH to 7 using 1N hydrochloric acid and 1N sodium hydroxide and adding water to 500 ml. The solution was homogenized by stirring and filtered through a sterile 0.22 μm filter, after which the solution was freezed into droplets of 0.1 ml, which droplets were transferred in the frozen state into a freeze dryer and then freeze-dried to unloaded spherical lyophilized dosage units (lyospheres).

120 mg of 5-chloro-2-methyl-2,3,3a, 12b-tetrahydro-1H-dibenz[2,3:6,7]oxepino[4,5-c]pyrrole maleate (1:1) (Org 5222) were dissolved in 1 ml of ethanol and 83 .mu.l of this solution were added to one lyospheres, after which the ethanol was removed by gentle heating, to obtain a lyosphere containing 10 mg of Org 5222. Lyospheres containing 1 and 0.1 mg of Org 5222 respectively, were prepared in a similar manner by dissolving 60 or 6 mg of Org 5222 respectively in 1 ml of ethanol, after which 16.6 μl of this solution were added to one lyosphere.

### Example 7

A pharmaceutical composition was prepared comprising:

0.094 g of 5-chloro-2-methyl-2,3,3a, 12b-tetrahydro-1H-dibenz[2,3:6,7]oxepino[4,5-c]pyrrole maleate (1:1) (Org 5222), 30 g of mannitol, 40 g of gelatin, and distilled water to a volume of 1 l, which composition was freeze-dried according to the method of Example 1b into unit dosage forms, each of which comprises 10 μg of Org 5222.

### Example 8

### Randomized, Placebo-Controlled, Double-Blind Trial Demonstrating the Safety and Efficacy of Sublingual Asenapine in In-Patients with an Acute Manic Episode

A trial for the indication acute manic and mixed episode of bipolar I disorder is performed (400-500 subjects).

The primary objective of the trial is to demonstrate Safety and Efficacy of Sublingual Asenapine vs. Placebo in change from baseline in the Young-Mania Rating Scale (Y-MRS) scores in subjects with manic or mixed episodes associated with bipolar I disorder.

Secondary objectives include evaluating treatment effects of asenapine with placebo with respect to:
changes in the Clinical Global Impressions Scale for use in Bipolar Disorder (CGI-BP) scores
changes in the Montgomery-Asberg Depression Rating Scale (MADRS) scores
changes in PANSS scores
safety and tolerability.

Trial medication includes active and placebo fast-dissolve asenapine tablets. Asenapine and placebo fast-dissolve tablets, will be identical in appearance and will be administered in a double-dummy fashion.

The trial is a 3-week, randomized, placebo-controlled, double-blind, double-dummy, multicenter, parallel-group trial. Subjects are randomly assigned to asenapine or placebo treatment.

The trial includes (up to) a 7-day single-blind placebo washout period during which subjects experiencing a manic or mixed episode receive single blind placebo. This period allows for washout of excluded medications and for receipt of clinical laboratory results.

The active treatment period is initiated on Day 1 with placebo or asenapine 10 mg BID. Thereafter, treatment is continued with flexibly dosed asenapine (5-10 mg BID) or placebo. Subjects must remain confined to an inpatient research facility for at least 7 days (through Day 7), but may be subsequently discharged if deemed clinically stable by the investigator.

The effect can be observed in one or more of the following measurements:

The change from baseline, last observation carried forward (LOCF), to Week 3 on the Y-MRS, the percent Y-MRS responders and remitters, the change from baseline on CGI-BP, MADRS, PANSS subscales (Marder positive, negative, disorganized thought, hostility/excitement, and anxiety/depression symptom scores. Efficacy scales can be analyzed at all assessed time points.

Asenapine can be evaluated for safety and tolerability compared with placebo during 3-weeks of exposure.

Assessments are described below:

### Efficacy:

Y-MRS: A 11-item, clinician-rated instrument for assessing the symptoms of mania
CGI-BP: A 7-point clinician-rated scale for assessing the severity and change from preceding phase of illness of manic, depressive, and overall symptoms of bipolar disorder during the treatment of an acute episode or in longer-term illness prophylaxis.
PANSS: A 30-item clinician-rated instrument for assessing psychotic or schizophrenic symptoms
MADRS: A 10-item clinician-rated scale for assessing the severity of symptoms of depression
Safety will be assessed through: Concomitant medication use, adverse events (AEs), weight, vital signs (heart rate, blood pressure, and respiration), physical exam, electrocardiogram (ECG), and clinical laboratory findings (hematology, biochemistry, and urinalysis) and scores on the 3 scales used to assess EPS: Barnes Akathisia Rating Scale (BARS), Abnormal Involuntary Movement Scale (AIMS), and Simpson Angus Rating Scale (SARS).

Follow-up safety assessments: Subjects who discontinue their participation in the trial either prematurely or complete the acute trial but do not continue in the extension trial are contacted 7 days after the End of Treatment (EOT) visit for follow-up on any ongoing AEs or serious AEs (SAEs). Patients will be contacted 30 days after EOT to document any additional SAEs.

1. A method for treating or preventing mania or hypomania associated with a bipolar disorder in a mammal in need thereof comprising administering to said mammal a formulation comprising a pharmaceutically effective amount of a compound of Formula I as the only pharmaceutically active compound present therein, 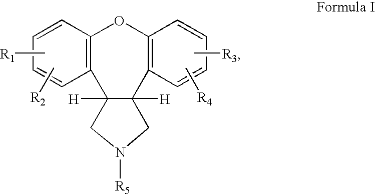
or a pharmaceutically acceptable salt, solvate, hydrate or optical isomer thereof, wherein R₁, R₂, R₃, and R₄ represent a member selected from the group consisting of hydrogen, hydroxy, halogen, C₁-C₆ alkyl, C₁-C₆ alkoxy, C₁-C₆ alkylthio, and trifluoromethyl; and R₅ represents hydrogen, C₁-C₆ alkyl or aralkyl having from 7 to 10 carbon atoms.

2. The method of claim 1 wherein the compound of Formula I used in the Formulation is trans-5-chloro-2-methyl-2,3,3a, 12b-tetrahydro-1H-dibenz[2,3:6,7]oxepino[4,5-c]pyrrole (asenapine), which has a structure of the compound of Formula Ia 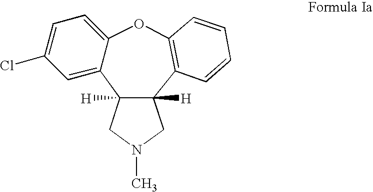
or a pharmaceutically acceptable salt thereof.

3. The method of claim 2 wherein the symptom of bipolar disorder treated is mania.

4. The method of claim 2 wherein the symptom of bipolar disorder treated is hypomania.

5. A method of stabilizing mood in a mammal afflicted with bipolar disorder, which method comprises administering to said mammal formulation comprising a pharmaceutically effective amount of a compound of Formula Ia 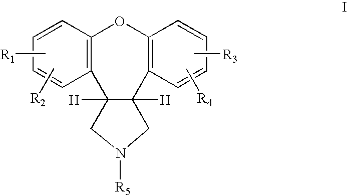
or a pharmaceutically acceptable salt thereof as the only pharmaceutically active compound present in the formulation.

6. The method of claim 5 wherein the compound of Formula I is asenapine or a pharmaceutically acceptable salt thereof.

7. A method for treating bipolar disorder and at least one other co-morbid or concomitant disease, condition or disorder selected from depression, melancholia, fatigue, personality disorders including avoidant personality disorder, borderline personality disorder, schizotypal personality disorder, and anxious personality disorder, aggressive disorders including intermittent explosive disorder and organic personality syndrome, oppositional defiant disorder, atypical cycloid psychoses, motility psychosis, confessional psychosis, anxiety-blissfulness psychosis, dementia or delirium in a mammal in need thereof comprising administering to said mammal a pharmaceutically effective amount of a formulation comprising asenapine or a pharmaceutically acceptable salt thereof, as the only pharmaceutically active agent.

8. The method of claim 1 wherein the compound is trans-5-chloro-2-methyl-2,3,3a, 12b-tetrahydro-1H-dibenz[2,3:6,7]oxepino[4,5-c]pyrrole and is administered in dosages of about 0.5 mg to about 500 mg per day.

9. The method of claim 2 wherein the compound is trans-5-chloro-2-methyl-2,3,3a, 12b-tetrahydro-1H-dibenz[2,3:6,7]oxepino[4,5-c]pyrrole and is administered in dosages of about 0.5 mg to about 500 mg per day.

10. The method of claim 8 wherein the formulation is administrated sublingually, buccally, or supralingually.

11. The method of claim 9 wherein the formulation is administrated sublingually, buccally, or supralingually.

12. The method of claim 1 wherein the treatments effect improvement in the mammal within about 96 hours after administering the compound.

13. The method of claim 2 wherein the treatments effect improvement in the mammal within about 96 hours after administering the compound.

14. The method of claim 1 wherein the treatments effect improvement in the mammal within about 24 to about 96 hours after administering the compound.
